(19) United States
(12) Patent Application Publication
(10) Pub. No.: US 2014/0045185 A1
(43) Pub. Date: Feb. 13, 2014

(54) Novel Gene Normalization Methods

(71) Applicant: Stokes Bio Limited, Limerick (IE)

(72) Inventors: Mark DAVIES, Limerick (IE); Tara DALTON, Limerick (IE)

(73) Assignee: Stokes Bio Limited, Limerick (IE)

(21) Appl. No.: 13/963,253

(22) Filed: Aug. 9, 2013

Related U.S. Application Data
(63) Continuation of application No. 12/539,773, filed on Aug. 12, 2009, now abandoned.
(60) Provisional application No. 61/088,134, filed on Aug. 12, 2008.

(51) Int. Cl. C12Q 1/68

(52) U.S. Cl. CPC C12Q 1/6886
USPC 435/6.12

(57) ABSTRACT

Measurement of gene expression relative to an endogenous control gene is prone to excessive variability between samples and even replicates. The disclosure provides methods for normalizing expression levels of a gene by scaling gene expression levels to that of the most highly expressed gene in the set of genes whose expression levels are measured, rather than a house-keeping gene.

### RELATED APPLICATIONS

The present application claims priority to U.S. Provisional Application No. 61/088,134, filed Aug. 12, 2008, the contents of which are incorporated by reference.

### TECHNICAL FIELD

The invention is in the field of molecular biology and relates to methods for gene expression and biomarker analysis, including using diagnostic measurements using quantitative polymerase chain reaction (qPCR).

### BACKGROUND

Gene expression signatures comprised of tens of genes have been found to be predictive of disease type and patient response to therapy, and have been informative in countless experiments exploring biological mechanisms. For interpretation of quantitative gene expression measurements in clinical tumor samples, a normalizer is necessary to correct expression data for differences in cellular input, RNA quality, and RT efficiency between samples. In many studies, a single house-keeping gene is used for normalization. Conventionally, gene expression is normalized to an endogenous control gene. The endogenous control gene should exhibit constant expression in all samples being compared. Usually, cellular maintenance genes, the so-called house-keeping genes, are selected to normalize for the variability between clinical samples. These genes regulate basic and ubiquitous cellular functions and code, for example, for components of the cytoskeleton (β-actin), major histocompatibility complex (e.g., β-2-microglobulin), glycolytic pathway (e.g., glyceraldehyde-3-phosphate dehydrogenase (GAPDH) and phosphoglycerokinase 1), metabolic salvage of nucleotides (e.g., hypoxanthine ribosyltransferase), protein folding (e.g., cyclophilin), or synthesis of ribosome subunits (e.g., rRNA). In many experiments, the expression of these genes is assumed invariable between cells of different samples and used as normalizer without proper validation. However, there is no universal control gene expressed at a constant level under all conditions and in all tissues. For instance, cellular RNA content as well as expression levels of house-keeping genes may vary due to a disease (e.g., malignancies) or other cellular condition resulting in inaccurate normalization, and therefore inadequate quantification and spurious conclusions.

As an illustration, the acute leukemias are broadly classified into those that arise from the lymphoid precursors (acute lymphoblastic leukemias; ALL) and those that arise from myeloid precursors (acute myeloid leukemia; AML). ALL can be divided into several subtypes by molecular and cytogenetic techniques. The use of gene expression as a diagnostic for types and subtypes of leukemia has been severely limited given the inherent imprecision of microarray systems and normalization of data to an endogenous control leading to erroneous results (Perez et al. (2007) BMC Molecular Biology, 8:114). The selection of a small number of statistically significant genes from microarray data (van Delft et al. (2005) British Journal of Haematology, 130:26-35) has permitted the use of qRT-PCR to be performed instead, which allows more accurate and precise gene expression measurement. However, measurements of gene expression relative to an endogenous control gene are still prone to excessive variability between samples and even replicates.

Therefore, there exists a need for new methods of gene normalization that are less prone to uncertainty when compared to endogenous control, in general, and more specifically, for classifying the types and sub-types of diseases (e.g., cancers) in a clinical diagnosis.

### SUMMARY OF THE INVENTION

The present invention provides novel methods of normalizing gene expression levels. Expression levels are usually normalized per total amount of RNA or protein in the sample and/or an endogenous control gene, which is typically a house-keeping gene such as, e.g., actin or GAPDH). This invention is based, at least in part, on the discovery that normalization to the highest expressed gene is less prone to uncertainty of endogenous control normalization. In the experiments described here, expression data were compared for 96 genes in six independent leukemic cell lines cultured in vitro. These cell lines are known to carry either an acute lymphoblastic leukemia (ALL) or acute myeloid leukemia (AML) type translocations. Additionally, DNA from 21 patient samples was blind tested for which the subtype was previously diagnosed. A method for diagnosing the sub-types of paediatric leukemia is thereby proposed and can be employed to accurately discriminate the subtypes within both types of childhood leukemia. Furthermore, the normalization method may be broadly applied in any setting where gene expression is evaluated. The methods of the invention described can be used in any method that requires evaluation of gene expression levels of one or more genes.

Accordingly, the invention provides novel methods of evaluating gene expression levels. Methods of the invention include:

a) determining expression levels of a plurality of genes in a biological sample under substantially similar conditions,

b) scaling the expression levels relative to the highest expressed gene in the plurality of genes, said highest expressed gene being other than a house-keeping gene; and

c) evaluating the scaled expression levels of one or more of the genes.

Biological samples used in the methods of the invention may be obtained from a subject's bodily fluid or tissue, or from a cell line or tissue culture. In some embodiments, the gene expression measurements of multiple genes are performed in separate replicates of a sample individually and/or expression levels of a gene may be measured in replicates. The gene expression levels may be determined at the RNA or the protein level. In preferred embodiments, the measurements are performed using the polymerase chain reaction (PCR), particularly, quantitative PCR (qPCR).

In some embodiments, the evaluated genes include biomarkers of a disease or condition. In further embodiments, the methods of the invention are used for diagnosing a subject, including gene expression profiling. The invention also includes methods for identifying and/or validating biomarkers which may be used in the diagnostic methods. In illustrative embodiments, the methods of the invention are used to diagnose subtypes of childhood leukemia, such as ALL and AML.

Additional aspects of the invention are described in detail below.

### BRIEF DESCRIPTION OF THE FIGURES

FIG. 1 depicts the maximal-inclusive scaling (MIS) method applied to the ALL and AML biomarker set. The first three samples (0412005Fujioka-Stokes, Fujoika-Barts, and PatientE) are three samples with AML. Discrimination between and AML and other samples is clear, especially, with respect to Gene 5.

FIG. 2 represents clustering of ALL (left) and AML (right) samples using MIS.

FIG. 3 represents a comparison of two normalization methods for the gene sets and samples shown in FIG. 2. FIG. 3a shows normalization to a house-keeping gene (GAPDH). FIG. 3b shows normalization to the maximally expressed gene in a subset. Solid lines represent AML samples.

### DETAILED DESCRIPTION OF THE INVENTION

The invention provides novel methods of evaluating gene expression levels. Methods of the invention include:

a) determining expression levels of a plurality of genes in a biological sample under substantially similar conditions;

b) scaling the expression levels relative to the highest expressed gene in the plurality of genes, said highest expressed gene being other than a house-keeping gene; and

c) evaluating the scaled expression levels of one or more of the genes.

A plurality of genes may include 2, 3, 4, 5, 10, 25, 50, 100 or more genes. In some embodiments, the mostly highly expressed gene is expressed at levels that are at least 10%, 20%, 30%, 50%, 2×, 3× or higher than the closest highly expressed gene. The most highly expressed gene may be a biomarker of disease of condition

### Expression Levels

Expression levels, at the RNA or at the protein level, can be determined using any suitable methods, including many currently available conventional methods. RNA levels may be determined by, e.g., quantitative PCR (e.g., TaqMan™ PCR or RT-PCR), Northern blotting, or any other method for determining RNA levels, e.g., as described in Sambrook et al. (eds.) Cloning: A Laboratory Manual, 2nd ed., Cold Spring Harbor Laboratory Press, 1989, or as described in the Examples. Other amplification methods can also be used, including the ligase chain reaction (LCR), the transcription based amplification system (TAS), the nucleic acid sequence-based amplification (NASBA), the strand displacement amplification (SDA), rolling circle amplification (RCA), hyper-branched RCA (HRCA), etc. In preferred embodiments, the measurements are performed at the RNA level using the qPCR.

Numerous target-specific probes are available from commercial sources. A desired set of probes may also be synthetically made using conventional nucleic acid synthesis techniques. For example, probes may be synthesized on an automated DNA synthesizer using standard chemistries, such as, e.g., phosphoramidite chemistry.

Protein levels may be determined, e.g., by using Western blotting, ELISA, enzymatic activity assays, or any other method for determining protein levels, e.g., as described in Current Protocols in Molecular Biology (Ausubel et al. (eds.) New York: John Wiley and Sons, 1998).

### House-keeping Genes

The invention involves the use of the mostly highly expressed gene in a subset, as an endogenous control. In certain embodiments, such a gene is not a house-keeping gene. House-keeping genes are constitutively expressed to maintain cellular function. As such, they are presumed to produce the minimally essential transcripts necessary for normal cellular physiology. With the advent of microarray technology, it has recently become possible to identify at least the “starter set” of house-keeping genes, as exemplified by the work of Velculescu et al. (1999) “Analysis of human transcriptomes” Nat. Genet. 23:387-388, as well as by Warrington et al. (2000) Physiol. Genomics 2:143-147, in a paper published in this journal previously. In that paper, Warrington et al. examined the expression of 7,000 full-length genes in 11 different human tissues, both adult and fetal, to determine the suite of transcripts that were commonly expressed throughout human development and in different tissues. The authors identified 535 transcripts via microarray hybridization as likely candidates for house-keeping genes, or “maintenance”, genes. Additional examples of house-keeping genes can be found in Hsiao et al. (2001) “A compendium of gene expression in normal human tissues” Physiol. Genomics, 7:97-104; and Eisenberg (2003) “Human House-keeping genes are compact” published in Trends in Genetics 19:362-365 (see also www.compugen.co.il/supp_info/House-keeping_genes.html). Select examples of house-keeping genes are illustrated in Table 1.

TABLE 1
Select examples of house-keeping genes

| Gene name | Abbreviation | Cellular function |
|---|---|---|
| Large ribosomal protein | LRP | Transcription |
| β-actin | BACT | Cytoskeleton |
| Cyclophilin A | CYC | Serine-threonine |
| | | phosphatase inhibitor |
| Glyceraldehyde-3- | GAPDH | Glycolysis enzyme |
| phosphate dehydrogenase | | |
| Phosphoglycerokinase 1 | PGK | Glycolysis enzyme |
| β-2-microglobulin | B2M | Major histocompatibility |
| | | complex |
| β-glucuronidase | BGUS | Exoglycosidase in lysosomes |
| Hypoxanthine | HPRT | Metabolic salvage of purines |
| ribosyltransferase | | |
| TATA-box-binding protein | TBP | Transcription by RNA |
| | | polymerases |
| Transferrin receptor | TfR | Cellular iron uptake |
| Porphobilinogen deaminase | PBGD | Heme synthesis |
| ATP synthase 6 | ATP6 | Oxydative phosphorylation |
| 18S ribosomal RNA | rRNA | Ribosome subunit |

### Biological Samples

Methods of the invention involve analysis of gene expression levels in a biological sample. A biological sample may contain material obtained cells or tissues, e.g., a cell or tissue lysate or extract. Extract may contain material enriched in sub-cellular elements such as that from the Golgi complex, mitochondria, lysosomes, the endoplasmic reticulum, cell membrane, and cytoskeleton, etc. In some embodiments, the biological sample contains materials obtained from a single cell.

Biological samples can come from a variety of sources. For examples, biological samples may be obtained from whole organisms, organs, tissues, or cells from different stages of development, differentiation, or disease state, and from different species (human and non-human, including bacteria and virus). The samples may represent different treatment conditions (e.g., test compounds from a chemical library), tissue or cell types, or source (e.g., blood, urine, cerebrospinal fluid, seminal fluid, saliva, sputum, stool), etc.

Various methods for extraction of nucleic acids from biological samples are known (see, e.g., Nucleic Acids Isolation Methods, Bowein (ed.), American Scientific Publishers, 2002). Typically, genomic DNA is obtained from nuclear extracts that are subjected to mechanical shearing to generate random long fragments. For example, genomic DNA may be extracted from tissue or cells using a Qiagen DNeasy Blood & Tissue Kit following the manufacturer's protocols.

In some embodiments, the biological sample is derived from a cell line, optionally, treated with an agent whose effect on gene expression is evaluated. In other embodiments, the sample is a tissue or a biological fluid of a subject (e.g., a mammal, (e.g., a rodent or a primate, e.g., human)).

In some embodiments, the biological sample is divided into replicates (e.g., duplicates, triplicates, etc.) in which the expression levels are measured. The sample may be derived from the same source and split into replicates just prior to measuring the expression levels. Replicate samples may be analyzed in a serial or parallel manner. Gene expression levels for the same gene may be measured in replicates, and the final gene expression level expressed as an average or a mean of the replicates, or an otherwise calculated level representing multiple samples. In some embodiments, expression levels of two or more genes are measured in separate replicates individually. Alternatively, or in addition, the expression levels of at least some genes may be measured in the same reaction volume, e.g., using multiplex PCR.

### Biomarkers

In some embodiments, a plurality of genes being measured comprises at least one biomarker of a disease, including a disease type or subtype. As used herein, the term “disease” includes a pathologic or otherwise abnormal condition identifiable by altered gene expression levels. As used herein, a biomarker is a gene whose expression correlates with the presence of a specified disease or condition. Such a disease or condition may be due to a pathogen, e.g., virus, fungus, bacteria, or a toxin. A disease or condition may be of any type, e.g., malignancy, immunological disorder, cardiovascular, or neurological. For example, cancers being evaluated may include, for example, cancers of colon, breast, prostate, skin, bladder, or lung as well as lymphoma, leukemia, etc. Numerous biomarkers for various diseases and conditions are known (see, e.g., Biomarkers in Breast Cancer (Cancer Drug Discovery and Development), Humana Press; 1 edition, 2005); Biomarkers of Disease: An Evidence-Based Approach; Cambridge University Press; 1 edition, 2002). In illustrative embodiments, the cancer markers used are of pediatric leukemia, including the markers that allow differentiation acute lymphoblastic leukemia (ALL) or acute myeloid leukemia (AML) types, and further subtypes as illustrated in Table 3 and 4.

Thus, in some embodiments, methods of the invention are used for differentiation between disease types or subtypes by evaluating two or more biomarkers specific to one or more disease types or subtypes. For example, the methods may include evaluation of 2, 3, 4, 5, 10, 25, 50, 100 or more biomarkers of disease types or subtypes.

### Biomarker Selection

In additional aspects, the invention provides methods of selecting, identifying, or otherwise confirming a gene as a biomarker of a disease or pathological condition. The methods include:

a) determining expression levels of a first set of genes in a biological sample characterized by the presence of disease or a disease subtype;

b) determining expression levels of a second set of genes in a biological sample devoid of the disease or the disease subtype under substantially similar conditions as in a);

c) scaling the expression levels of genes in the first and second levels relative to the highest expressed biomarker in both sets, said highest expressed gene being other than a house-keeping gene; and

d) selecting one or more genes whose scaled expression level correlates with the presence of the disease or pathological condition, thereby identifying the gene(s) as a biomarker of the disease.

### Diagnostics, Prognostics, Testing, and Treatment Monitoring

The invention further provides methods for diagnosis or prognosis of disease or condition. The method comprising evaluating gene expression levels, by methods of the invention, in a biological sample obtained from a subject. The term “diagnosis” and its cognates, as used herein, include both diagnostic and prognostic methods. More specifically, such methods include:

a) determining expression levels of a plurality of genes in a biological sample obtained from a subject,

b) scaling the expression levels relative to the highest expressed gene in the plurality of genes, said highest expressed gene being other than a house-keeping gene; and

c) evaluating the scaled expression levels of one or more of the genes, thereby diagnosing the subject.

Methods of the invention may also be used, for example, for evaluating a treatment administered to a subject or the course of evaluating the efficacy or toxicity of a drug. In some of these embodiments, a biological sample being evaluated is obtained from cells or an animal treated with such a drug.

The following Example provides illustrative embodiments of the invention and does not in any way limit the invention.

### EXAMPLES

Samples—Complementary DNA (cDNA) from the various cell lines is obtained from the following cell lines MHHCALL, SD1, REH, 697 and MOLT 4I which represent the ALL type, and the Fujioka cell line which represents the AML type. cDNA sample are obtained from patients who were previously diagnosed with the ALL and AML types and subtypes.

TABLE 2
Model cell lines and corresponding translocations/karyotypes for ALL or
AML types of leukemia

| Type | Subtype | Karyotype | Model cell line |
|---|---|---|---|
| ALL | Hyperdiploid (HD) | More than two copies of a | MHH CALL |
| | | chromosome | SD1 |
| ALL | BCR-ABL | t(9; 22) | SDI |
| ALL | ETV6-RUNX1 | t(12; 21) | REH |
| ALL | E2A-PBX1 | t(1; 19) | 697 |
| ALL | T-cell ALL | | MOLT4 |
| AML | CALM-AF10 | t(10; 11) | Fujioka, U937 |

Quantitative RT-PCR—The TaqMan® Immune Profiling Low-Density Array consists of 96 TaqMan® gene expression assays (Applied Biosystems) preconfigured in 384-well format and spotted on a microfluidic card (4 replicates per assay). Each TaqMan® gene expression assay consists of a forward and reverse primer at a final concentration of 900 nM and a TaqMan® MGB probe (6-FAM dye-labeled; Applied Biosystems), 250 nM concentration. The assays are gene-specific and are designed so that they span an exon-exon junction. Each assay and its ID number are available from www3.appliedbiosystems.com/cms/groups/mcb marketing/documents/generaldocuments/cms 040290.pdf. First, 350 μl of cDNA from each cell line sample and patient sample are combined in an Eppendorf® tube with an equal volume of TaqMan® Universal qRT-PCR mastermix (Applied Biosystems). The contents of the eppendorf is mixed by inversion, and spun briefly in a microcentrifuge. Once the cards had reached room temperature, 100 μl of each sample is loaded into each of the eight ports on the TaqMan® low-density array. The cards are placed in a Sorvall/Heraeus custom buckets (Applied Biosystems) and centrifuged in a Sorvall Legend™ Centrifuge for one minute at 331 g. Cards which exhibited excess sample in the fill reservoir are spun for an additional one minute. Following centrifugation the cards are immediately sealed using a TaqMan® Low Density Array sealer (Applied Biosystems) to prevent cross-contamination. The final volume in each well following centrifugation is less than 1.5 μl. The qRT-PCR amplifications are conducted on the ABI 7900HT real-time PCR system. The thermal cycling conditions used are as follows: 10 min at 95° C. (activation), 50 cycles of denaturation at 97° C. for 30 s, and annealing and extension at 59.7° C. for 1 minute. Independent cell lines and patient samples are run on separate cards.

Analysis—The following analysis considers the measured expression levels of the 96-well assay of biomarkers derived from the larger set in (van Delft et al., supra). The analysis presented here considers 59 biomarker genes, as the remaining the remaining genes are endogenous controls or biomarkers associated with subtypes not to be classified here.

The set of biomarkers is subdivided into subsets of markers for the types and subtypes using a gene array hybridization technique presented in (van Delft et al., 2005). A summary of the type and subtype subsets is given in Table 3 with the number of genes in each set. Note that only four genes are included in each of the ALL and AML subsets. These should allow type discrimination while the other subsets should allow subtype discrimination. This work focuses on ALL/AML discrimination, ALL subtype discrimination, and MLL (a subtype of AML) discrimination. For validation purposes, the gene expression data is obtained from qRT-PCR experiments which are conducted in two locations (Stokes Institute in Limerick, and St. Bartholomew's Hospital, London) for six distinct cell lines, and 21 distinct patient samples, all with three replicates to each processed card (see Table 4). Partial least squares in conjunction with entropy-based discretization may be used to predict the diagnosis of unknown samples. The efficacy of this approach may be investigated by use of leave-one-out cross validation, allowing estimation of false negative rates and false positive rates. Finally, the scaling method implemented here may be compared to normalization relative to an endogenous reference.

TABLE 3
Biomarker gene sets associated with specific subtypes.

| ALL sets | | AML sets | |
|---|---|---|---|
| Type of | | Type of | |
| subtype | Genes | subtype | Genes |
| ALL | 4 | AML | 4 |
| Hyperdiploid (HD) | 27 | MLL | 5 |
| BCR-ABL | 15 | | |
| ETV6-RUNX1 | 3 | | |
| E2A-PBX1 | 2 | | |
| AML1 | 6 | | |
| T-ALL | 2 | | |

TABLE 4
Types and subtypes for cell lines/patient samples and corresponding
number of cards processed.

| Cell line | Type | Subtype | Cards |
|---|---|---|---|
| REH | ALL | ETV6-RUNX1 | 3 |
| SD1 | | BCR-ABL & HD | 2 |
| MHHCALL | | HD | 2 |
| 697 | | E2A-PBX1 | 2 |
| MOLT4 | | T-ALL | 1 |
| Fujioka | AML | | 2 |
| Patient samples | Type | Subtype | Cards |
| | ALL | ETV6-RUNX1 | 3 |
| | | T-ALL (& MLL) | 1 |
| | | T-ALL (only) | 2 |
| | | E2A-PBX1 | 3 |
| | | AML1 | 4 |
| | | BCR-ABL | 3 |
| | | HD | 2 |
| | AML | MLL | 1 |
| | | Not MLL | 3 |

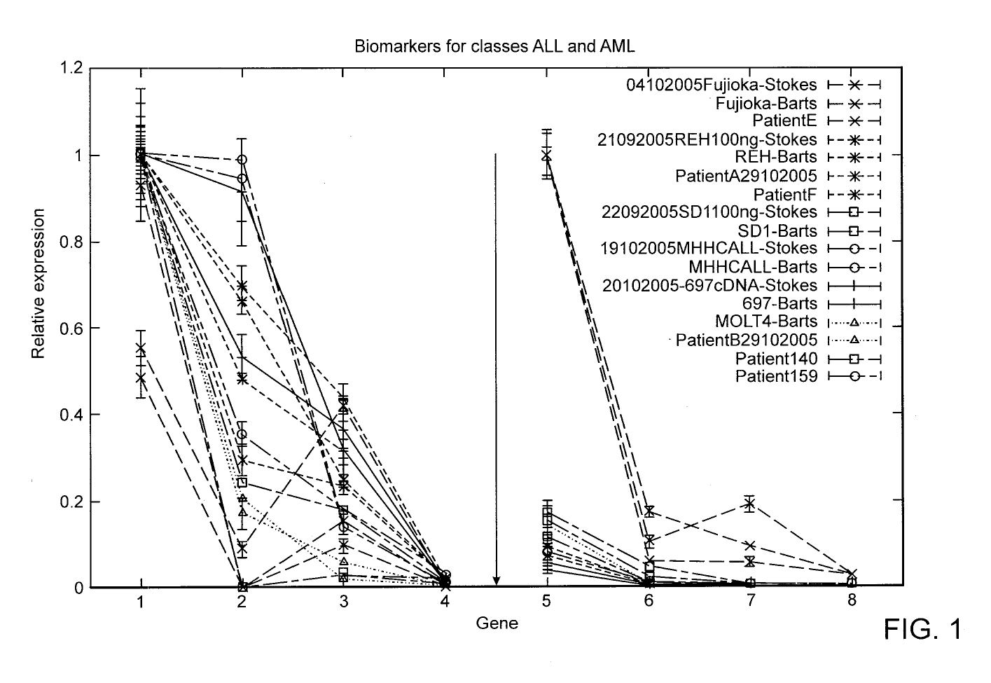

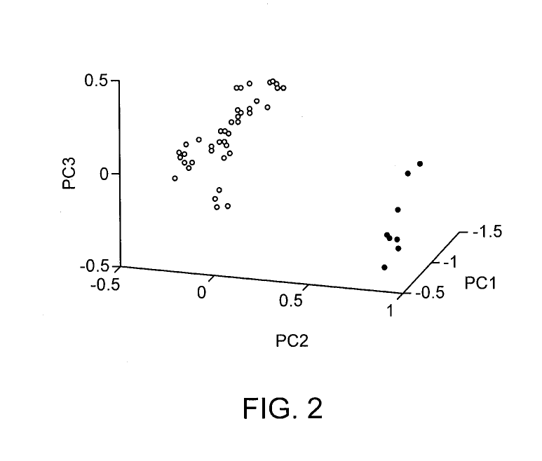

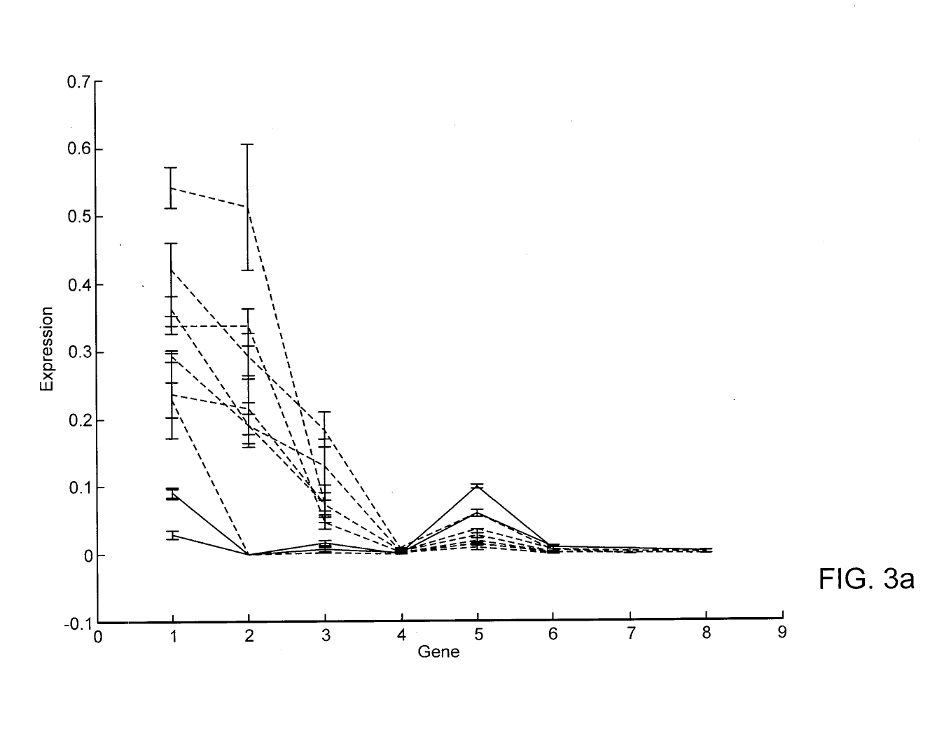

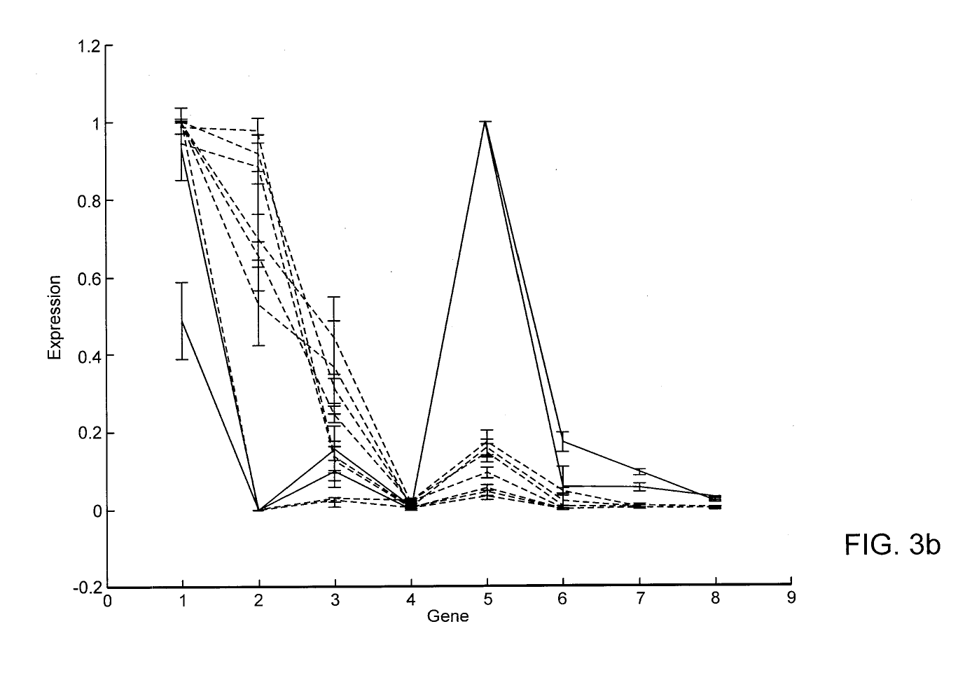

Maximal Inclusive Scaling—Maximal inclusive scaling (MIS) refers to the normalization of gene expression data, as described here, as an alternative to normalization relative to a endogenous control gene. The steps are generally as follows:
Choose two types/sub-types (classes): Class A and B
The expression of biomarker genes in class A are {Aᵢ} and those in class B are {Bᵢ}
For any given example (card replicate) find the highest expression among the genes, max{Aᵢ,Bᵢ}
Scale expression of all genes {Aᵢ,Bᵢ} relative to max{Aᵢ,Bᵢ}.
   The resulting expression measurement for the genes in the set {Aᵢ,Bᵢ} are now relative to the maximally expressed gene and not relative to the endogenous gene. FIG. 1 represents a plot of the MIS process applied to a number of samples. In this case the classes A and B are ALL and AML, respectively, and distinction between these classes os possible by a qualitative inspection of the relative values. It is clear from the data that both {Aᵢ} and {Bᵢ} are markers for both types. For ALL samples, A₁ is the most expressed among {Aᵢ,Bᵢ}, A₂ & A₃, and B₁≦0.2. In contrast, for AML samples, max{Aᵢ,Bᵢ}=B₁ and A₃>A₂. Using singular value decomposition (SVD) (Wall et al., 2003), each replicate vector {Ai,Bi} may be projected onto a three dimensional space preserving as much variance as possible as shown in FIG. 2. Two separate clusters of datapoints are visible, one cluster associated with ALL (on the left) and the other, with AML (on the right). Alternatively, if gene expression is normalized by the endogenous control, these two clusters are no longer separate but instead overlap with each other.

Partial Least Squares for Classification—Singular value decomposition retains the structure of the measured gene expression profile by maximizing the variance explained in the reduced space. However, this does not necessarily provide the best discrimination in the reduced space. Partial least squares (PLS) (Boulesteix et al. (2007) Briefings in Bioinformatics, 8(1):32-44; Nguyen et al. (2002) Bioinformatics 18:39-50; Bastien et al. (2005) Computational Statistics & Data Analysis, 48:17-46; Gidskehaug et al. (2006) Chemometrics and Intelligent Laboratory Systems, 84(1-2):172-176) is a method that incorporates into the analysis the classification of the gene expression profile and is thus a supervised technique. Consider n observed examples of the expression of p genes. In this context, the class of the example is termed a response and the measured gene expressions are termed predictors as it is these values that allow prediction of the response. Matrix X of observations forms the matrix of predictors. Here, only univariate PLS is considered so that the response for each example is a scalar. Briefly, the PLS regression involves a decomposition of the predictor matrix X and the response matrix Y whose rows form the response vectors corresponding to the predictors. This can be summarized as follows:

X_{(n×p)}=T_{(n×c)}P^{T}_{(p×c)}+E_{(n×p)}, (1a)

Y_{(n×q)}=T_{(n×c)}Q_{(q×c)}^{T}+F_{(n×p)}, (1b)

where T is a n×c matrix of latent components for the n observations, P and Q are matrices of coefficients, and E and F are matrices of random errors.

In PLS, the latent components are constructed as a linear transformation of X

T=XW, (2)

where W is the matrix of weights. This may be combined with Eq. (1b) to yield the matrix of regression coefficients B

Y=TQ^{T}=XWQ^{T}=XB,

where B=WQ^{T}. Using B and given a gene expression profile x, the response y may be predicted to be

y=xB.

The response space in the classifications that are considered here is one-dimensional and real. For classification problems a predictor vector represents a sample that is either class or non-class: the response space is discrete. To classify an unknown sample the predicted response space must be discretized by partitioning it into class and non-class subsets at a particular threshold. One method to partition this space is to apply entropy-based discretization (Perner and Trautzsch (1998) “Multi-interval discretization methods for decision tree learning” in Advances in Pattern Recognition, S:475-482; Fayyad et al (1993) Proc. of the Thirteenth Int'l Joint Conference on Artificial Intelligence, 1022-1027; Ross et al. (2003) Blood, 102(8):2951-2959.

With a set of N cards, the predictive power of a classification may be estimated by using N−1 (training) cards to form B using PLS, and the threshold using entropy based discretization. One may then attempt to predict the class of the remaining (test) card, which has three gene expression profiles with three corresponding responses. This process may be repeated by assigning each of the N cards as a test card. The number of false positives Fₚ and the number of false negatives Fₙ allow estimation of the false positive rates

$$( α = \frac{{F}_{p}}{{N}_{n}} ;$$

FPR) and false negative rates

$$( β = \frac{{F}_{n}}{{N}_{p}} ;$$

FNR), where Nₚ is the number of positive (class) instances and Nₙ is the number of negative (non-class) instances. Estimates of the false negative and false positive rates are indicative of whether the classification method has potential as an aid to diagnosis.

TABLE 5
Table of estimated β and total false rate (TFR = α + β) using MIS
and PLS, upon performing leave one out cross validation.

| Couple | Test Class | ^{c}min | β | TFR |
|---|---|---|---|---|
| ALL + AML | AML | 2 | 0.00 | 0.00 |
| HD + T-ALL | AML1 | 2 | 0.00 | 0.03 |
| AML + BCR-ABL | BCR-ABL | 18 | 0.07 | 0.11 |
| AML + E2A-PBX1 | E2A-PBX1 | 2 | 0.00 | 0.00 |
| AML + ETV6-RUNX1 | ETV6-RUNX1 | 2 | 0.00 | 0.04 |
| AML + HD | HD | 7 | 0.06 | 0.09 |
| BCR-ABL + MLL | MLL | 3 | 0.17 | 0.34 |
| HD + T-ALL | T-ALL | 2 | 0.00 | 0.00 |

Table 5 shows values of β and TFR for a number of couples that demonstrate the best classification abilities for each subtype classification. The two subtype classifications which show poor performance are for the MLL and BCR-ABL subtypes. The poor performance of the MLL classification here may be attributed to the fact that only two cards of this class were available for training the PLS regression. However, the BCR-ABL subtype is a heterogeneous leukemic subtype reflected by the number of factors necessary for best classification being almost all factors (18) out of a possible 19.

All publications, patents, patent applications, and biological sequences cited in this disclosure ₐre incorporated by reference in their entirety.

1. A method of evaluating gene expression levels, the method comprising:
a) determining expression levels of a plurality of genes in a biological sample under substantially similar conditions,
b) scaling the expression levels relative to the highest expressed gene in the plurality of genes, said highest expressed gene being other than a house-keeping gene; and
c) evaluating the scaled expression levels of one or more of the genes.

2. The methods of claim 1, wherein the biological sample is divided into replicates in which the expression levels are measured.

3. The method of claim 2, wherein the expression levels of at least one gene measured in two or more replicates, and the expression levels of the gene is determined as an average or a mean of the replicates.

4. The method of claim 2, wherein the expression levels of two or more genes are measured in separate replicates individually.

5. The method of claim 1, wherein the plurality of genes comprises three or more genes.

6. The method of claim 1, wherein the scaled expression levels relative to the highest expressed gene more accurately represents relative expression levels of the genes than expression levels of the same genes normalized to an endogenous house-keeping gene.

7. The method of claim 1, wherein the gene expression levels are determined by PCR.

8. The method of claim 7, wherein the gene expression levels are determined by quantitative PCR.

9. The method of claim 8, wherein the biological sample is derived from a cell line, optionally, treated with an agent whose effect on gene expression is evaluated.

10. The method of claim 1, wherein the plurality of genes comprises at least one biomarker of a disease or condition.

11. The method of claim 1, wherein the disease or condition is due to a pathogen.

12. The method of claim 10, wherein the disease or condition is a cancer type or subtype.

13. The method of claim 11, wherein the cancer is leukemia.

14. The method of claim 1, wherein the plurality of genes comprises two biomarkers, each specific to a disease, condition, or disease or condition type or subtype.

15. The method of claim 11, wherein the cancer subtypes are ALL or AML.

16. A method for in vitro diagnosis, the method comprising evaluating gene expression levels using the method of claim 10, wherein the biological sample is obtained from a subject, thereby diagnosing the subject.

17. A method of identifying a biomarker of a disease or pathological condition, the method comprising:
a) determining expression levels of a first set of genes in a biological sample having a disease or a disease subtype;
b) determining expression levels of a second set of genes in a biological sample devoid of the disease or the disease subtype under substantially similar conditions as in a);
c) scaling the expression levels of genes in the first and second levels relative to the highest expressed biomarker in both sets, said highest expressed gene being other than a house-keeping gene; and
d) selecting a gene whose scaled expression level correlates with the presence of the disease or pathological condition, thereby identifying the gene as a biomarker of the disease.

18. A method for in vitro diagnosis, the method comprising evaluating gene expression levels using the method of claim 14, wherein the biological sample is obtained from a subject, thereby diagnosing the subject.
